(19) United States
(12) Patent Application Publication
(10) Pub. No.: US 2017/0218445 A1
(43) Pub. Date: Aug. 3, 2017

(54) DRUG DISCOVERY METHODS USING PLANT DEVELOPMENTAL BIOLOGY

(71) Applicant: SEOUL NATIONAL UNIVERSITY R&DB FOUNDATION, Seoul (KR)

(72) Inventors: Sunghwa CHOE, Seoul (KR); Maya Maharjan PUNA, Seoul (KR); Slki PARK, Seoul (KR); Richard CHA, Seoul (KR)

(73) Assignee: SEOUL NATIONAL UNIVERSITY R&DB FOUNDATION, Seoul (KR)

(21) Appl. No.: 15/319,581

(22) PCT Filed: Jun. 17, 2015
(86) PCT No.: PCT/KR2015/006118

§ 371 Date: Apr. 6, 2017

(30) Foreign Application Priority Data
Jun. 17, 2014 (KR) 10-2014-0073880

(51) Int. Cl. C12Q 1/68; G01N 33/96

(52) U.S. Cl. CPC C12Q 1/6876; C12Q 1/6897; G01N 33/96; C12Q 2600/13; C12Q 2600/136; G01N 2333/415

(57) ABSTRACT

The present disclosure relates to a method for discovering effective bioactive compounds, based on plant developmental biology, and aims to discover a lead bioactive compound for new drugs by repeating screening based on phenotypes of a plant as a marker on the basis of plant developmental biology. To this end, the present disclosure provides a method for discovering effective bioactive compounds, and includes screening candidates for developing a new drug by using unique phenotypes as a marker shown by a plant when it is grown with a specific material.

### FIELD

The present disclosure relates to a method for discovering effective bioactive compounds, based on plant developmental biology, to reduce costs and enhance safety in screening candidates for developing new drugs. More specifically, the present disclosure relates to a method for separating effective ingredients by using specific responses of Arabidopsis thaliana as a marker shown when various crude extracts and fractions thereof are applied to grow Arabidopsis thaliana seedlings.

### BACKGROUND

Plants have been used as drugs for treating human diseases for a long time. This is because bioactive small-molecule made by the plants can be used to treat human diseases.

From a modern perspective, developing a new drug is subject to a process of investing astronomical costs and then applying the drug to humans at the final stage to verify the effect thereof. However, in the traditional herbal medicine, effects and side effects of plants have been established empirically by using plants to humans directly.

As described above, although the effects of treatment with natural products including traditional herbal medicines have been established through experiences for a long time, one traditional herbal medicine contains hundreds to thousands of compounds. Therefore, it is inevitable to accept that it contains both medicinal ingredients and toxic ingredients. In particular, where the content of toxic ingredients of a drug is more than the lethal dose although it contains medicinal ingredients, it cannot be used for human diseases treatment.

Furthermore, it is highly likely that traditional herbal medicinal materials contain a very small amount of medicinal ingredients, respectively. In this regard, although a target ingredient is successfully discovered, the issue of smooth supply of crude drugs can be a challenge for commercialization thereof.

Therefore, in the aspect of new drug development, it is preferred in many ways to lower the complexity of active bioactive compounds in the traditional herbal medicines, or separate and use them to be in the level of a single bioactive compound. However, drug development is implemented very slowly because there is no available efficacy screening system, or efficacy screening systems are very expensive.

Meanwhile, it is essential to conduct repetitive testing of many medicinal properties in the process of separating active ingredients from crude extracts to be single bioactive compounds by testing their medicinal properties. Testing medicinal properties for separating active compounds varies from measurement of enzyme activity and cell testing to animal testing. However, one requirement of these methods is compliance with various regulations.

One of the conventional methods for identifying the most exact medicinal properties is animal testing. However, using mouse models of human disease to separate ingredients with medicinal properties from crude extracts costs tens of millions of Korean won, and the development cost to reach the stage just before marketing amounts to hundreds of billions of Korean won. Therefore, many researchers give up drug development even without making any attempt because of the required costs.

Because of the cost-related challenge, a large scale of capital investment is required to separate effective ingredients from traditional herbal medicinal materials. However, another issue is a high ratio of no formal approval because the value as a new drug is not certified at the final stage of clinical test despite the large scale of investment. That is, the possibility of successful new drug development is very low. The reason for unsuccessful drug development is based on high toxicity of new drug candidates, a low level of medicinal properties and too many side effects thereof.

Therefore, it is essential to discover bioactive compounds with less side effects and toxicity at greatly reduced costs. Although it is inevitable to pay the costs for direct test to humans which is a final stage and compulsory in developing a new drug, it is necessary to reduce as much cost as possible at stages before test on humans.

The basic biology of humans and plants has been conserved. Such a biochemical identity enables enzymes to mediate cell survival. By the way, the enzymes carry out almost the same function in many processes, for example, cell division, chromosome replication, RNA synthesis, protein metabolism, glycolysis, amino acid synthesis, small RNA metabolism, mitochondrial energy metabolism, etc.

Therefore, separation of active ingredients for traditional herbal medicines will allow researchers to use a plant system before animal testing or instead of animal testing. Development of a new drug at least targeting common metabolic pathways based on biology will allow preferential application of the plant system.

### SUMMARY

In view of the above, the present disclosure is based on the idea that the basic biology of humans and plants is conserved, and most genes or proteins targeted by drugs including anti-cancer drugs exist in plants, and aims to screen plants instead of animal testing.

Specifically, the present disclosure aims to discover a lead bioactive compound for a new drug through repetitive screening based on the expression of phenotypes as a marker in developmental biology of plants. In particular, the present disclosure aims to separate lead bioactive compounds from traditional herbal medicinal materials for new drugs.

Furthermore, the present disclosure aims to distinguish and standardize traditional herbal medicinal materials in a more quantitative and scientific way based on their medicinal properties.

Moreover, the present disclosure aims to discover bioactive compounds controlling plant growth.

### Technical Solution

In accordance with an embodiment of the present disclosure, there is provided a method for discovering effective bioactive compounds, based on plant developmental biology. The method includes screening candidates for developing a new drug or herbicide by establishing unique phenotypes shown in a plant as a marker when the plant is grown in media containing a specific compound. In this regard, the plant is preferably Arabidopsis thaliana (A. thaliana), of wild type, mutant and transgenic plants. Moreover, the transgenic plant can be a plant genetically modified by introducing chimeric genes which combine any one of genes of targeted human diseases, human cancer-causing genes, cosmetic genes, herbicide-resistant genes, and other genes having approved functions with reporter genes.

Furthermore, the present disclosure provides a method for standardizing effective traditional herbal medicinal materials, based on plant developmental biology. The method includes establishing unique phenotypes shown in a plant as a standard when the plant is grown in media containing an extract of traditional herbal medicinal material to grade traditional herbal medicinal materials. In this regard, the plant is a transgenic plant genetically modified by introducing chimeric genes which combine a gene promoter responding sensitively to an extract of specific traditional herbal medicinal material with reporter genes. As used herein, the traditional herbal medicinal material can be graded by quantitatively analyzing the level of reporter gene expression induced by the traditional herbal medicinal material

The present disclosure can reduce costs and enhance safety in screening candidates for developing new drugs.

Moreover, the present disclosure can discover quickly and cost-effectively the ingredients with medicinal properties for a new natural product-based drug currently available in the market including traditional herbal medicinal extracts of which the medicinal properties have been proved, and facilitate finding their mode of action.

Moreover, the present disclosure can be used for proving safety and medicinal properties of new drugs.

Furthermore, the present disclosure can contribute to developing and commercializing varieties of high medicinal contents of specific natural products where the level of their bioactivity is identified. This can increase farmers' income in the end in the region where the varieties are produced.

Moreover, because the present disclosure is based fundamentally on understanding botany, the present disclosure can lead development of basic botany. That is, it is possible to understand how plants establish their survival strategy through response to new bioactive compounds.

Moreover, the present disclosure can be applied to standardizing traditional herbal medicinal materials. Standardization of the materials requires technical standards. However, although index ingredients of the traditional herbal medicinal materials are known, specific active ingredients thereof have not been defined making it impossible to device accurate standardization. After describing specific phenotypes of a plant in response to a specific traditional herbal medicinal material, the traditional herbal medicinal material can be graded based on the phenotypes. This process can contribute to establish a scientific system of traditional herbal medicinal materials and facilitate industrialization of traditional herbal medicines.

Furthermore, because the bioactive compounds selected in accordance with the present disclosure are effective for controlling plant growth, the present disclosure can contribute to better cultivation of crops. For example, where a discovered bioactive compound has an allelopathic effect for inhibiting plant growth, it can be developed as a herbicide to inhibit unnecessary weed growth.

Moreover, the present disclosure can contribute to developing new genetically-modified plants with desired functions to lead development of high-tech farming based particularly on plant factories across the agricultural sector.

### BRIEF DESCRIPTION OF THE DRAWINGS

FIG. 1A is a photograph showing the cotyledons, hypocotyl and roots of three-day old A. thaliana seedlings; 1B is a photograph showing the root tip and root hairs of A. thaliana plant; 1C is a photograph showing a adult A. thaliana plants grown in soil; and 1D is a photograph showing single adult A. thaliana plant;

FIG. 2 shows a process of discovering a bioactive compound based on phenotypes of a plant as a marker;

FIG. 3 shows a process of obtaining fractions from a crude extract;

FIG. 4 shows a screening method by using a genetically modified plant;

FIG. 5 shows a method for standardizing traditional herbal medicinal materials;

FIG. 6 shows a flowchart of understanding the mechanism of action of active ingredients;

FIG. 7 shows a graph illustrating optical absorbance of crude extracts analyzed in accordance with PC1 and PC2 of PCA (Principal Component Analysis);

FIG. 8 is a photograph showing response of A. thaliana seedling to taxol and camptothecin;

FIG. 9 is a photograph showing control roots and taxol-treated roots of A. thaliana seedlings;

FIG. 10 is a photograph showing the trichome of an A. thaliana seedling which changes from a three-branch type in wild type to a one-branch type in taxol treated seedling;

FIG. 11 shows a graph illustrating changes in hypocotyl length growth of A. thaliana seedlings depending on the concentrations of taxol and camptothecin;

FIG. 12 is a photograph showing response of A. thaliana seedling to hyulbuchukeo-tang and medicinal material components thereof;

FIG. 13 is a photograph showing response of A. thaliana seedling to Tao He Cheng Qi Tang and medicinal material components thereof;

FIG. 14 is a photograph showing response of A. thaliana seedlings to kyungheom-bang and medicinal material components thereof;

FIG. 15 is a photograph showing response of A. thaliana seedlings to other anticancer drugs (saengjihwang, yeongji, aeyeop, gyeowusari);

FIG. 16 shows a graph illustrating the number of lateral roots of A. thaliana seedlings in response to traditional herbal medicinal materials at 1/5× concentration, in which the symbol * represents statistical significance (n>8, p<0.05 Student's t-test);

FIG. 17 shows a graph illustrating the number of lateral roots of A. thaliana seedlings grown on the different concentrations of Tao He Cheng Qi Tang, daehwang and gyeji, in which the numbers on the horizontal axis represent dilution factors;

FIG. 18 is a photograph showing A. thaliana roots to illustrate response thereof to the concentrations of crude ginseng extract; and

FIG. 19 is a photograph showing an A. thaliana seedling of which the leaf petiole color changes to reddish brown in response to jacho.

### DETAILED DESCRIPTION

Hereinafter, the embodiments of the present disclosure will be described in detail.

1. Tested Plants

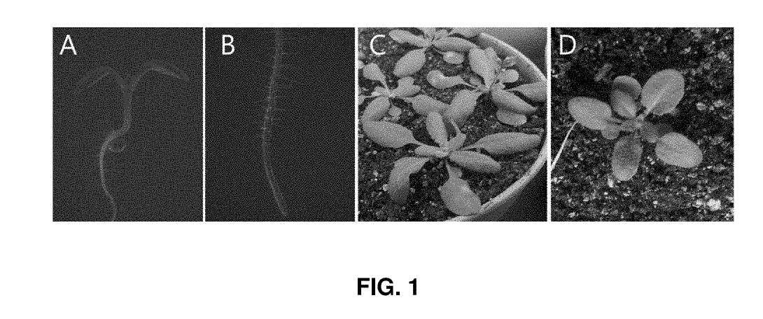

Arabidopsis thaliana (A. thaliana, shown in FIG. 1) which is a dicotyledon plant belonging to the Brassicaceae family has been studied by researchers in the field of plant developmental biology for the last 30 years to achieve great innovative discoveries (see www.arabidopsis.org). In particular, the reference genome sequence and the gene map of the A. thaliana published first in 2000 help researchers to understand the system biology thereof based on molecular genetics and genomics. A. thaliana is the first plant of which the reference genome sequence and the gene map were identified.

Studies of A. thaliana plants for many years have facilitated understanding overall growth, primary and secondary metabolisms, development of roots and stems, development of lateral roots, development of floral organs, development of leaves, flowering time regulation, hormone synthesis and signal transfer, and the mechanism to cope with environmental stress of A. thaliana plants in terms of genes thereof.

A. thaliana plants can be grown in a small space because their seeds are smaller than other plants, and A. thaliana seedlings are also very small. Because the A. thaliana seedlings are very small, it is possible to grow one seedling in each well of a 96-well plate to test the effect of any drug. This advantage allows HTS (High Throughput Screening).

Furthermore, their life cycle is very short. That is, it takes approximately four weeks to grow A. thaliana plants to adult stage and approximately six weeks to complete the life cycle. This is the shortest plant life cycle among seed plants. Therefore, this is an advantage for mass cultivation in a laboratory to observe changes in their growth over time.

Moreover, various types of cultivation techniques can be applied, for example, liquid and agar-solid media, soil and hydroponic cultivation depending on analysis requirements. The present disclosure is based on the process of germinating A. thaliana seeds followed by growing A. thaliana seedling in a liquid medium containing natural products for a given period, examining and analyzing their responses.

2. Method for Discovering Bioactive Compound Based on Phenotypes of A. thaliana as Marker

When phenotypes of a plant induced by the ingredients with medicinal properties are established, they can be used to discover bioactive compounds thereof.

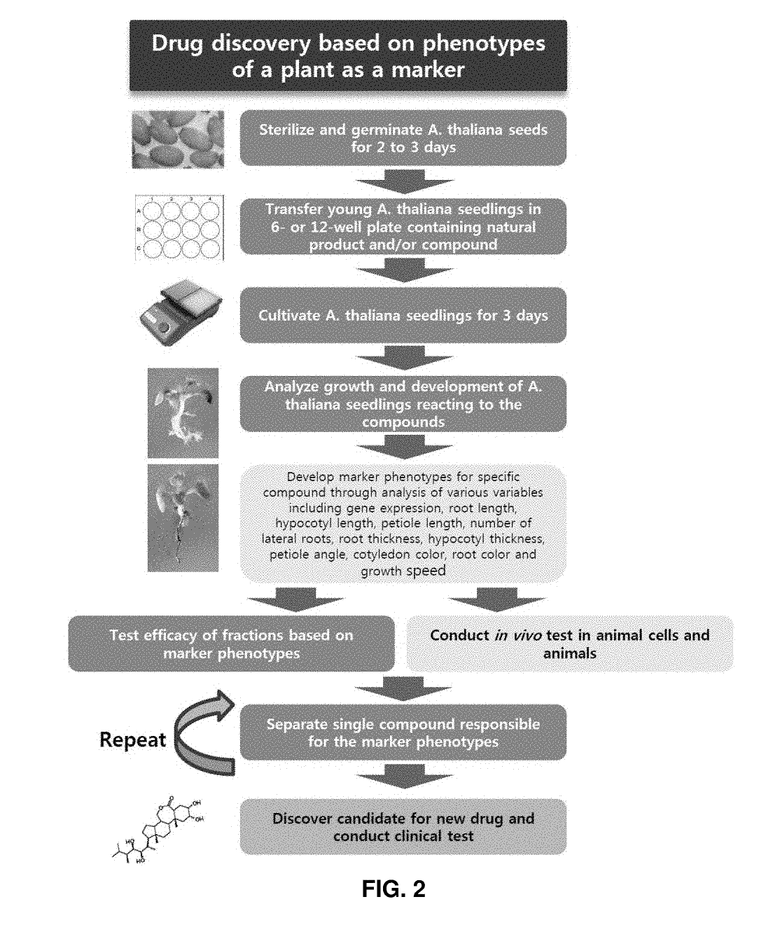

FIG. 2 shows a process of discovering a bioactive compound based on phenotypes of a plant as a marker. As shown in FIG. 2, the present disclosure concerns the method for discovering the ingredients with medicinal properties based on a specific growth response, that is, phenotypes as a marker, shown after growing a seedling in a medium containing a given natural bioactive compound or material. In this process, it is possible to separate a single bioactive compound that generates phenotypes as a marker by repetitively fractioning and testing the effects of a crude extract generating the phenotypes as a marker.

3. Separation of Bioactive Compound

The method for discovering a bioactive compound as a single ingredient from a crude extract or fractions is divided into two approaches.

The first approach is step-by-step fractioning.

This is a method for fractioning a crude extract systematically depending on solubility, molecular weight, etc. to conduct activity test. This method can reduce the complexity of medicinal properties of a natural product and separate single compounds of medicinal properties.

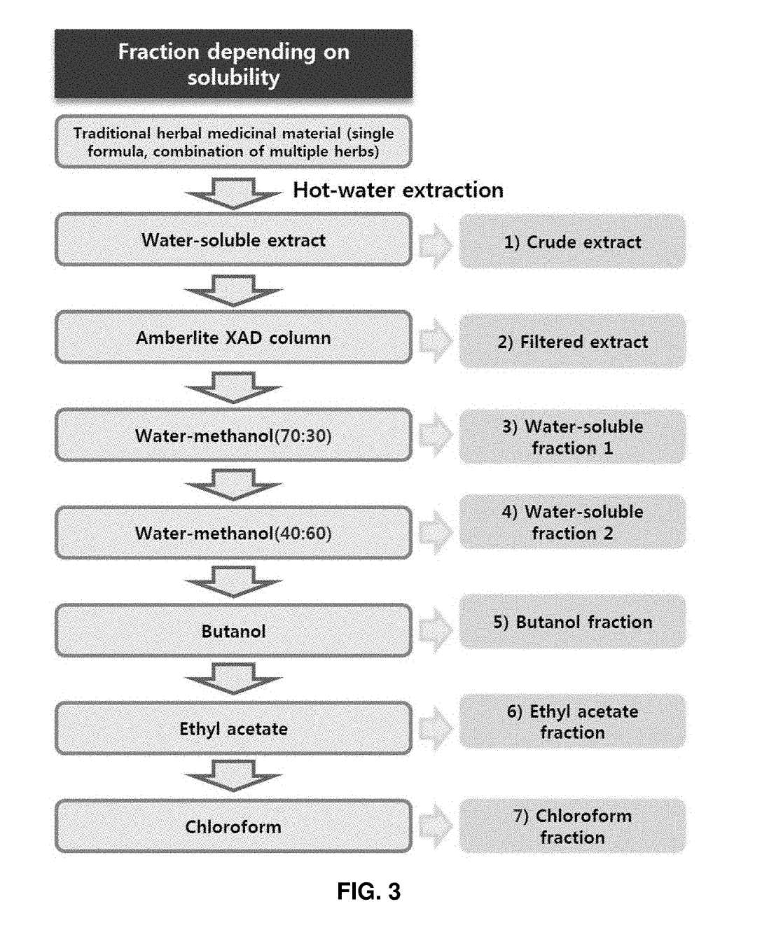

FIG. 3 shows a process of obtaining fractions from a crude extract based on solubility differences. As shown in FIG. 3, fractions are obtained while changing the solvent from a polar solvent (methanol) similar to water to a non-polar solvent (acetic acid or chloroform), and the bioactive compound dissolved in the solvent are then tested. Where a specific fraction shows a high activity, the fraction can be further separated depending on its molecular weight, etc. to test its activity and thus separate single bioactive compound.

The second approach is directly separating bioactive compounds absorbed into roots.

This is for separating whole bioactive small-molecule absorbed by roots treated with some specific natural product. For that purpose, bioactive compounds present in control roots and treated roots are compared and then analyze differences. The fractions are analyzed by FT-IR, measurement of broad optical absorbance, HPLC and MS/MS. When a difference is found in a specific bioactive compound, it can be subject to the primary analysis of active bioactive compounds.

4. Development of Automated Quantitative Analysis System Responding to Active Ingredients of Specific Natural Product

One of the impact of the technology in accordance with the present disclosure is the ability of providing a technology for standardizing traditional herbal medicinal materials. To this end, there is a need for developing and using a system for measuring the response of plants to natural products more quantitatively.

As an example, the following method can be used for quantitative comparison of reporter gene expressions. An exemplary process includes using a microarray or conduct RNA sequencing after treating A. thaliana seedling with an extract and then finding genes responding thereto. Next, a chimeric gene Promoter-Luciferase or Promoter-Venus (GFP) reporter system is developed, which combines the promoter of genes of which the expression to a specific extract is induced with a reporter gene. Such a reporter system can be applied to A. thaliana plants to produce a transformant and then use the plants for quantitative analysis.

This means that it is possible to identify the level of reporter gene expressions induced by a specific extract of medicinal material from each location or of development step. It is also possible to identify the level of expressions induced by each chemical fraction of the extract to measure quantity of a specific bioactive compound.

5. Identification of Mechanism of Action of Bioactive Ingredient

It is very important in many ways to understand the mechanism of action of a bioactive compound in the targeted gene level. The first significance is found in terms of botany. It is very interesting to study what defense mechanism a A. thaliana plant uses to react to an external small-molecule bioactive compound applied thereto just in terms of botany. It is because the result of the study can offer clues for understanding the mechanism about how a plant evolutionally reacts to its surrounding environment.

A traditional method based on genetics is used to discover such a mechanism. Approximately 200,000 EMS-treated mutant plants are grown in media to which a relevant natural product is added.

After growing them for a given period, resistant mutants which do not show specific phenotypes or sensitive mutants which show specific phenotypes as a marker can be separated. The gene causing the a mutant resistance to extract can be identified through whole genome resequencing after of the mutant after removing background mutations not related to the relevant phenotypes through several times back cross. Because the genome sequencing and functional genomics of A. thaliana as a plant model have been developed, it is easy to understand mechanisms thereof based on genetics.

It is also possible to establish the mechanism of action of humans. When a specific biological process is identified involved in the A. thaliana plants, this can be traced with human biology. As camptothecin does, it is possible to explain the mechanism focused on relevant genes involved in human cell divisions where the effect of action of topoisomerase I on plants is identified.

6. Screening Method by Using Genetically Modified Plant

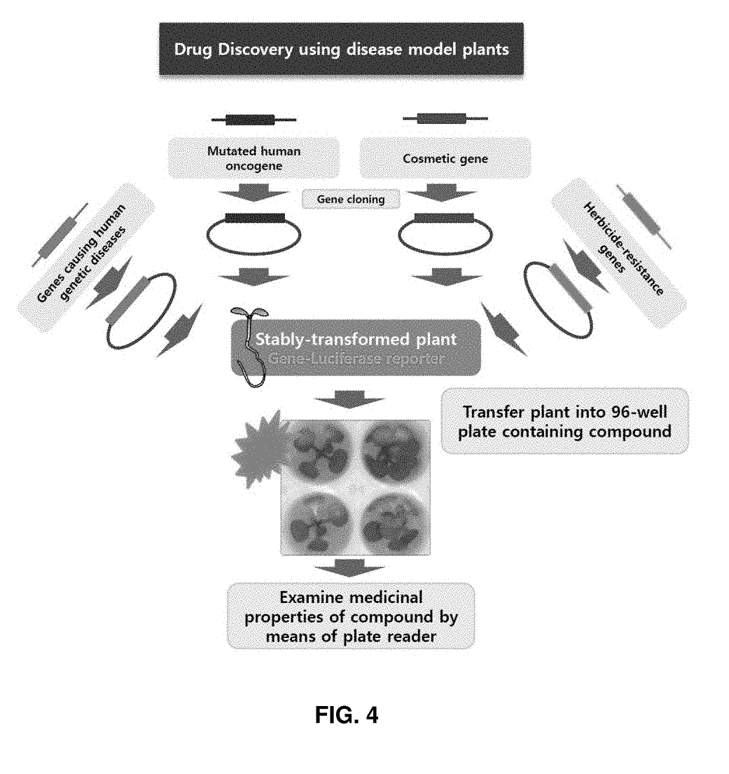

FIG. 4 shows a screening method by using a genetically modified plant. The genes of already known functions can be cloned and transformed into A. thaliana plants to produce transformants thereof, in addition to the genes of targeted human diseases, mutated human oncogenes, cosmetic genes, and genes resistant to herbicide, as shown in FIG. 4. These genes can be combined with reporter genes which makes the activity of bioactive compounds easily detectable through a photo-chemical process. For example, human disease genes can be combined with luciferase reporter genes to make chimeric genes. And transformants can be made by introducing these chimeric genes into plants. And then, various natural products can be applied to the transformants to examine and analyze their response quantitatively, by detecting luciferase activities.

7. Method for Standardizing Traditional Herbal Medicinal Material

Once plant phenotypes induced by the ingredients with medicinal properties are established, they can be used to standardize traditional herbal medicinal materials. Therefore, traditional herbal medicinal materials collected from different regions or at different times can be graded, focusing on the plant phenotypes, that is, on the basis of their medicinal properties.

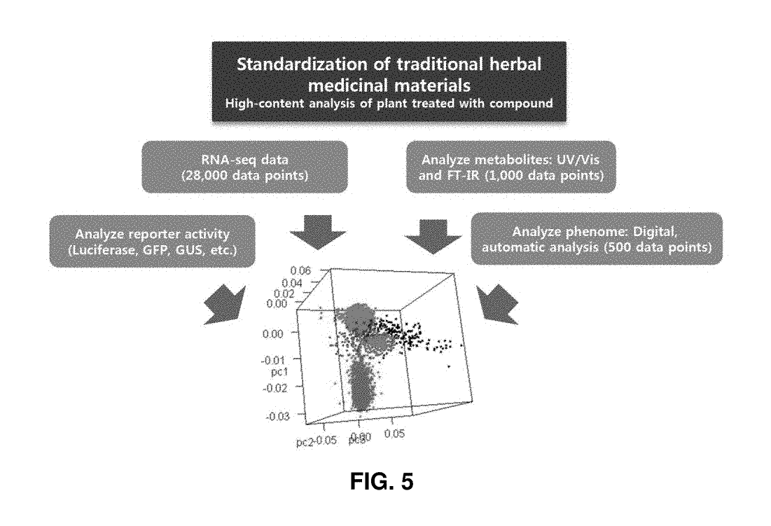

FIG. 5 shows a method for standardizing traditional herbal medicinal materials. It is possible to evaluate how much of active ingredients a medicinal material examined contains by comprehensively analyzing data, for example, reporter activities, phenotypes as a marker (phenome) reacting to a natural product, metabolite patterns, and RNA-seq data.

8. Understanding Mechanism of Action by Active Ingredients

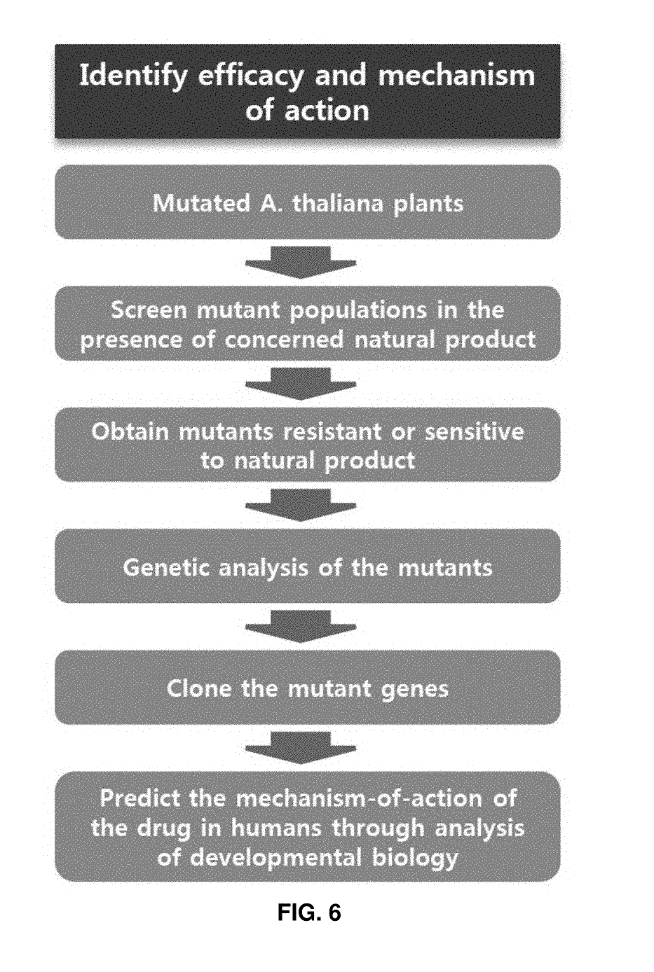

FIG. 6 shows a flowchart of understanding the mechanism of action of active ingredients. This flowchart is based on a process of finding genes targeted by an active bioactive compound by using genetics of A. thaliana plants and then understanding the process of biological action thereof. After understanding this process in plants, it is then possible to analogize and apply this process to human biology.

Exemplary Experiment

1. Preparing Hot-Water Extract of Traditional Herbal Medicinal Material

The hot-water extraction technique instead of using an organic solvent was applied in order to prepare an extract of which the medicinal properties of the material were proved in the conventional use thereof. The traditional herbal medicinal material was bought from a store in the Gyeongdong market, Jegi-dong.

First, the dried traditional herbal medicinal material was cut into small pieces to put them in a glass bottle together with 10 ml of distilled water per 1 g of the medicinal material. The glass jar was loosely closed with its cap and then placed in an autoclave to conduct extraction for 1.5 hours at 110° C.

After finishing extraction at the high temperature in the autoclave, the glass bottle was placed in an oven at 50° C. to cool it slowly. The extract was transferred into 50 ml-Falcon tubes And centrifuged at 2650 rpm for 10 minutes in order to remove solids residues and floating particles, and the supernatant liquid was then put into a new tube. The final extract obtained in the process described above was used as an undiluted solution (1×) to separate and test active ingredients in the following process.

2. Analysis of Optical Absorbance of Different Hot-Water Extracts

The optical absorbance of the hot-water extract of traditional herbal medicinal material was examined at different wavelengths to know whether hot-water extracts of the traditional herbal medicinal materials contain different natural products on a spectroscopic basis, and such a difference can be a proof showing one traditional herbal medicinal material can be distinguished from other traditional herbal medicinal materials.

The optical absorbance was measured in 3 replicates of extract in 6- or 12-well cell culture plate (SPL, Korea) diluted with distilled water and using the Epoch Microplate Spectrophotometer (BioTek, the US). The distilled water used in the above dilution was a control. The optical absorbance was measured at the intervals of 5 nm from 200 nm to 995 nm, and a statistical analysis was carried out using the values obtained by subtracting the optical absorbance of the distilled water from the optical absorbance of the extracts.

The R package was used for PCA (Principal Component Analysis) to show the difference among various traditional herbal medicinal materials in a 2-dimensional graph.

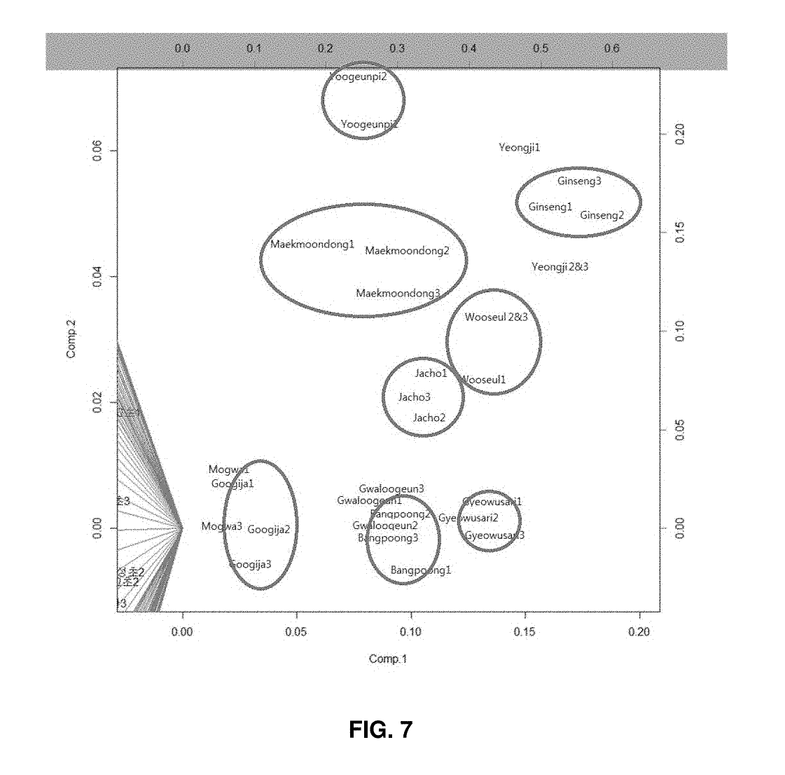

FIG. 7 shows a graph illustrating optical absorbance of crude extracts analyzed in accordance with PC1 and PC2 of PCA, in which the optical absorbance distribution of natural products based on the same traditional herbal medicinal material are shown relatively close. It is also shown that the optical absorbance of natural products based on different traditional herbal medicinal materials are distributed in different locations in the 2-dimensional space because of their difference in terms of their ingredient contents.

Therefore, this method can be applied to identifying unknown natural products, and can be used as a standardization tool for identifying true natural products or examining effective ingredient contents thereof depending on closeness of optical absorbance. That is, the aforementioned aspect of distribution can be used for standardizing the contents of medicinal ingredients or medicinal materials based on natural products.

3. Cultivating Young A. thaliana Plant

The Columbia seeds collected from wild-type A. thaliana plants were sterilized and then kept in a dark place at 4° C. for 3 days to enhance germination rate thereof. The wild-type A. thaliana seeds sown on an MS solid medium containing agar were germinated at 22° C. and on the long day condition of 16 hours the day/8 hours night and allowed to grow the A. thaliana seedlings for three days.

The undiluted extract of traditional herbal medicinal material described above was diluted at the ratios of 0.5×, 0.25× and 0.05×, and the MS liquid medium of 1 ml was mixed well with distilled water to make a total volume of 2 ml. in a 12-well cell culture plate. About ten A. thaliana seedlings were planted in each well and the plates were put on a shaker to cultivate them at 100 rpm for three days. Growth and development of the arabidopsis seedlings was analyzed after three days. Each test was carried out in 3 replicates.

The analysis of A. thaliana seedling response to anticancer drugs was made in the same manner, except that the anticancer drugs were applied instead of the traditional herbal medicinal material extract.

Root length, the number of lateral roots and hypocotyl length of the A. thaliana seedlings were measured in order to find the difference in developmental biology shown by the response of A. thaliana seedlings to the anticancer drugs and the extract. The ImageJ software (see rsb.info.nih.gov/ij/) was used to measure the respective lengths in images of the photograph file.

4. Response of A. thaliana Seedlings to FDA-Approved Aanticancer Drug

(1) Selecting Anticancer Drug

The reaction of A. thaliana plants to taxol (ingredient: Paclitaxel) and camptothecin, US FDA-approved plant-based anticancer drugs, was examined in order to verify whether the growth of A. thaliana seedlings can be used as phenotypesas a marker.

Taxol is produced in the plant in the genus Taxus, and is known for the effect of its action on tubulin to inhibit chromosome movement in cell division. In comparison with taxol, camptothecin is produced with barks and stems of Camptotheca acuminata, and is known for the effect of its action on DNA topoisomerase I to inhibit relaxation of DNA supercoiling. In other words, it was just determined that observation on the two anticancer drugs with different mechanisms of action generated different phenotypes in the seedlings could prove the advantage of the test method in accordance with the present disclosure.

(2) Change in Root Length and Number of Lateral Root of A. thaliana Seedling Depending on Concentrations of Taxol and Camptothecin

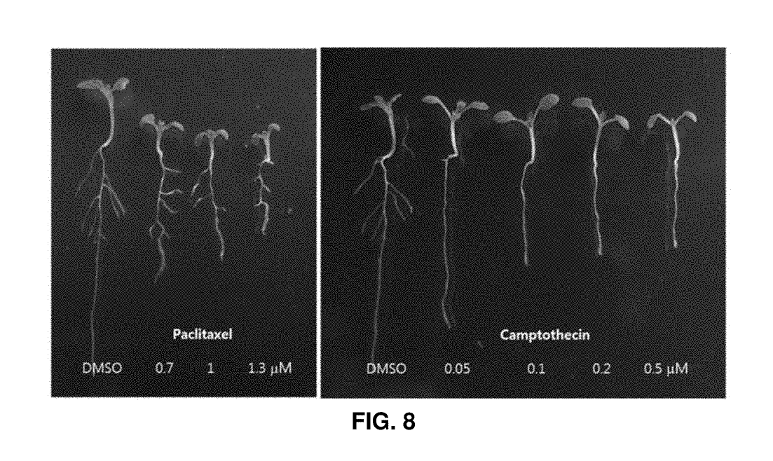

FIG. 8 is a photograph showing A. thaliana seedling treated with taxol and camptothecin, respectively.

As seen from FIG. 8, the root and hypocotyl growth of A. thaliana treated with taxol was inhibited in proportion to the concentrations thereof in comparison with the DMSO-treated control A. thaliana seedlings. However, generation of the lateral roots was not significantly affected. Approximately five lateral roots were generated whether the plants were treated or not with taxol.

In comparison with taxol, camptothecin markedly inhibited generation of the lateral roots. Camptothecin inhibited generation of lateral roots even at low concentrations, for example, 0.05 uM. As described above, it is seen that anticancer drugs with different mechanisms of action induce different growth patterns in plants. This suggests the possibility of screening effective bioactive compounds by using such a growth pattern as a marker.

(3) Changes in Root Morphology of A. thaliana Seedling Depending on Taxol Treatment

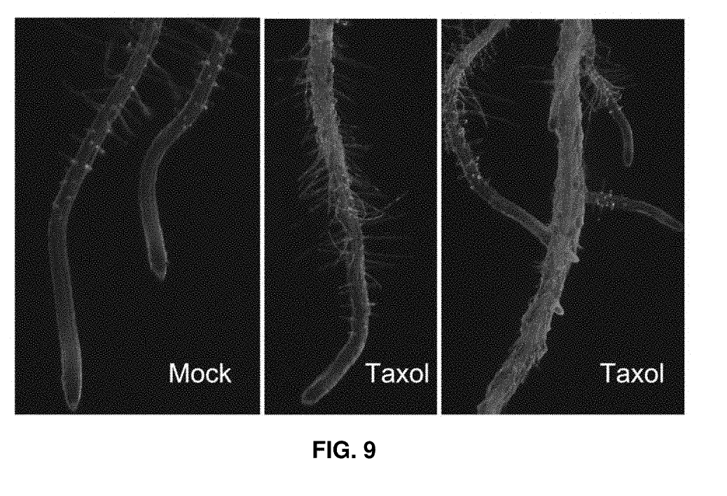

FIG. 9 is a photograph showing control roots and taxol-treated roots of A. thaliana seedlings. The photograph with the sign Mock shows control roots treated with water; and the photographs with the sign Taxol shows the roots three days after treatment with 2 uM of taxol. The roots treated with taxol have dense root hairs and opaque epidermal cells in comparison with those of the control roots. This is because the epidermal cells of roots are separated from tissues and have an atypical shape. This is a unique phenotype of taxol, suggesting that it guides apparent characteristics of plant growth which can be used as phenotype, that is, a marker.

(4) Effects of Taxol Treatment in Trichome Morphology of A. thaliana Seedling

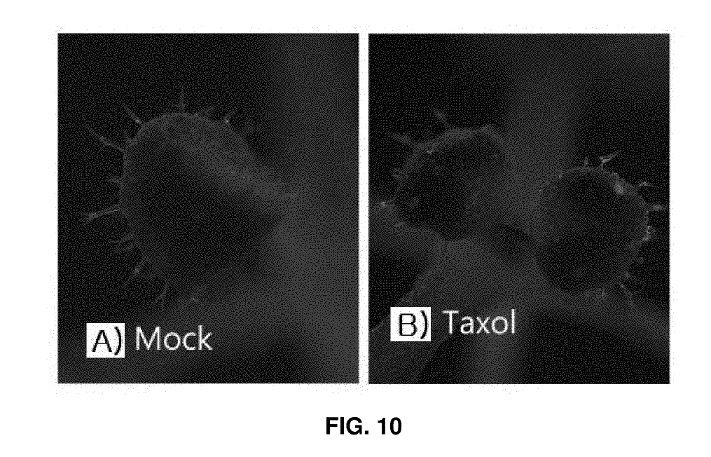

FIG. 10 is a photograph showing the trichome of an A. thaliana seedling which changes from a three-branch type in controlseedling to a one-branch type in taxol treated seedling.

Plants make numerous hairs on their leaf surface to protect themselves from insects, and A. thaliana seedlings have three-branched hairs. However, as shown in FIG. 10, the trichome branches changed from the three-branch type to the one-branch type in the A. thaliana plant treated with taxol in comparison with the control group. trichome development is closely related to root hair development, and the molecular mechanism thereof is well known.

(5) Changes in Hypocotyl Length of A. thaliana Seedling Depending on Concentrations of Taxol and Camptothecin

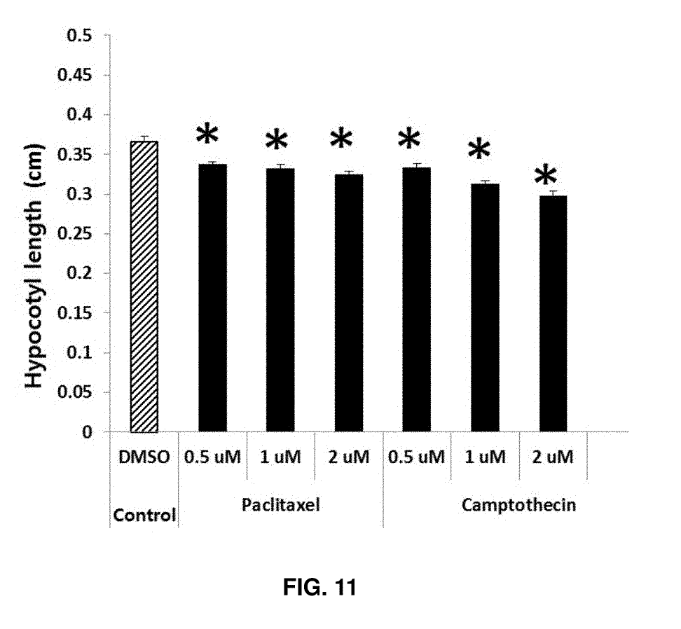

FIG. 11 shows a graph illustrating changes in hypocotyl length of A. thaliana seedling depending on the concentrations of taxol and camptothecin. Statistical computing was conducted to examine changes of hypocotyl growth depending on the concentrations of taxol and camptothecin. As seen in FIG. 11, overall hypocotyl growth was inhibited in a significant level.

5. Response of of A. thaliana Seedling to Traditional Herbal Medicinal Material

(1) Selecting Traditional Herbal Medicinal Material to be Examined

It was determined to examine traditional herbal medicinal materials with similar functions on the basis of the aforementioned response to the anticancer drugs. First, ‘hyulbuchukeo-tang’ (Moon, et al., 2006) and ‘Tao He Cheng Qi Tang’ generally known for their anticancer effects were selected from the traditional herbal prescription drugs. ‘Kyungheom-bang’ was also selected through inventor's experience of the present disclosure. First examination was made for the aforementioned three prescription drugs, their medicinal material components and some of other anticancer drugs.

(2) Prescription and Medicinal Material Components of Examined Traditional Herbal Medicinal Material

Following Table 1 illustrates prescription of the examined traditional herbal medicinal materials and medicinal material components thereof.

Crude extracts were made with the traditional herbal medicinal materials of each prescription and medicinal material components thereof, respectively, and the ⅕-fold-diluted solution was used as a first effective concentration. The solution was produced by adding 0.4 ml of the crude extract to 1 ml of the 0.5× MS medium and 0.6 ml of water.

TABLE 1

| | | | | Kyungheom-bang | |
|---|---|---|---|---|---|
| Hyulbuchukeo-tang | | Tao He Cheng Qi Tang | | Medicinal | |
| Medicinal material | | Medicinal material | | material | |
| component | Weight | component | Weight | component | Weight |
| Wooseul | 12 g | Daehwang | 15 g | Hagocho | 9 g |
| Danggui | 12 g | Doin | 9 g | Keumeunhwa | 9 g |
| Saengjihwang | 12 g | Mangcho | 9 g | Yeongyo | 9 g |
| Doin | 9 g | Gamcho | 9 g | Gookhwa | 8 g |
| Honghwa | 9 g | Gyeji | 9 g | Gwaruin | 5 g |
| Jeokjakyak | 9 g | Keumeunhwa | 8 g | Jinpi | 5 g |
| Siho | 9 g | Pogongyeong | 8 g | Yuhyang | 5 g |
| Cheongung | 6 g | | | Molyak | 5 g |
| Gilgyeong | 6 g | | | | |
| Gamcho | 3 g | | | | |

(3) Response of A. thaliana Seedlings to Crude Extract

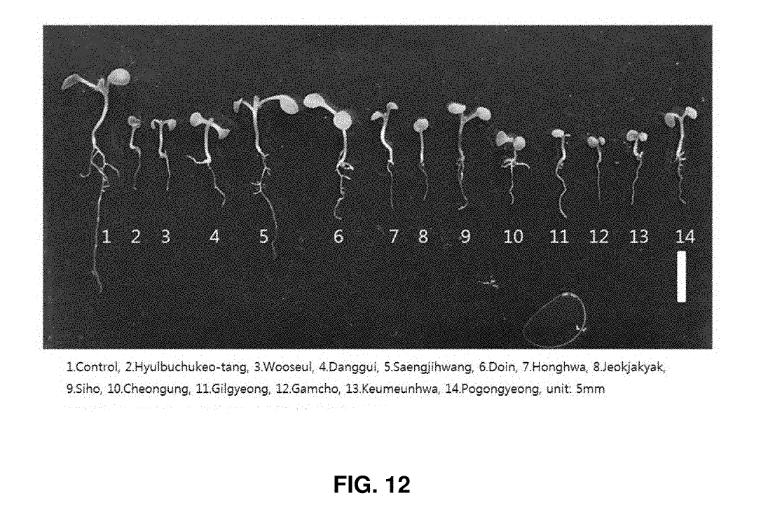

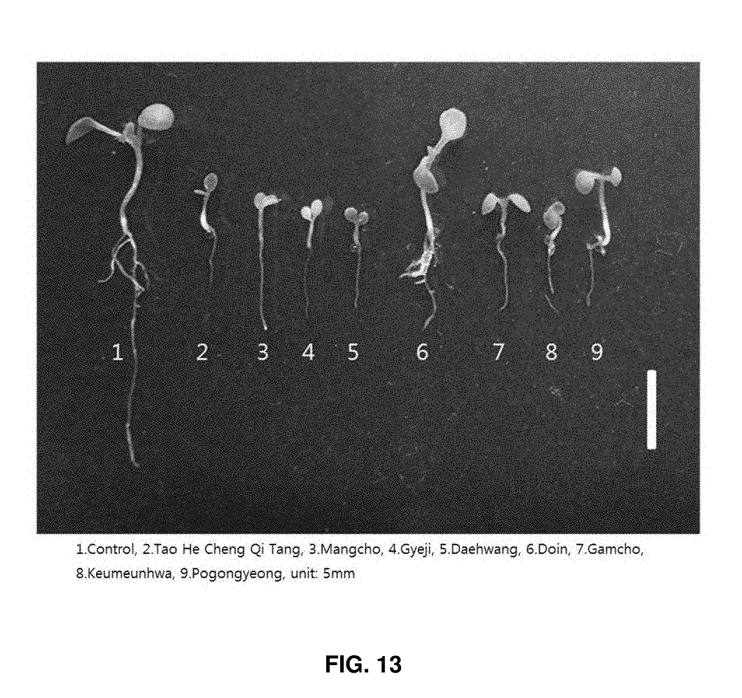

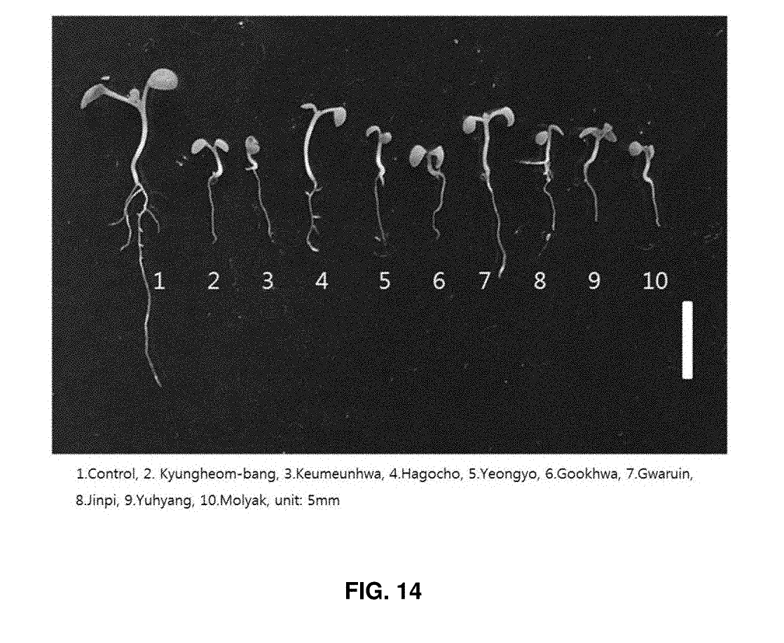

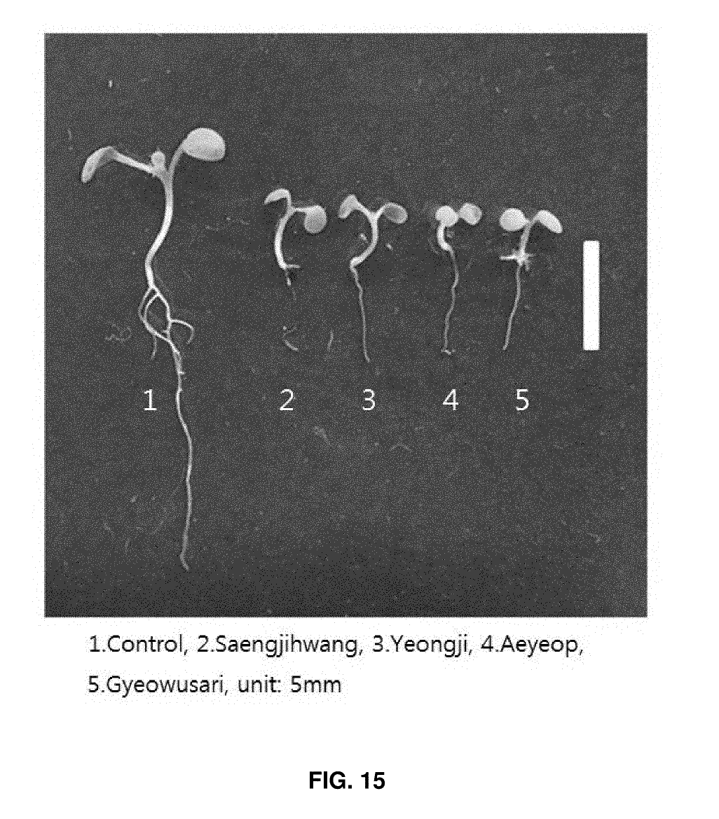

FIG. 12 is a photograph showing response of A. thaliana seedlings to hyulbuchukeo-tang and medicinal material components thereof. FIG. 13 is a photograph showing response of A. thaliana seedlings to Tao He Cheng Qi Tang and medicinal material components thereof. FIG. 14 is a photograph showing response of A. thaliana seedlings to kyungheom-bang and medicinal material components thereof, and FIG. 15 is a photograph showing response of A. thaliana seedlings to other anticancer drugs (saengjihwang, yeongji, aeyeop, gyeowusari).

As seen from FIGS. 12 to 15, the roots were generally shorter in length, and generation of lateral roots was inhibited. Moreover, some root color changed to brown. However, some medicinal materials were not strong in terms of inhibition of root growth, and hypocotyl growth in length was regulated in the same way as the root growth. That is, inhibition of root growth in length was observed along with inhibition of hypocotyl growth.

Considering that prescription drugs used as anticancer drugs in traditional herbal medicine and medicinal material components thereof generally inhibit generation of lateral roots, the possibility of separating new bioactive compounds is suggested by using the phenotypes inhibiting generation of lateral roots as a marker.

It is known that the lateral roots of A. thaliana plants are formed by repetitive division after the pericycle cells of roots obtain division capability (Dubrovsky, et al., 2000). Therefore, it is expected that the traditional herbal medicinal materials inhibiting generation and growth of lateral roots contain bioactive compounds generally inhibiting cell division.

(4) Statistical Analysis of Lateral Root Generation as a Response to the Extract

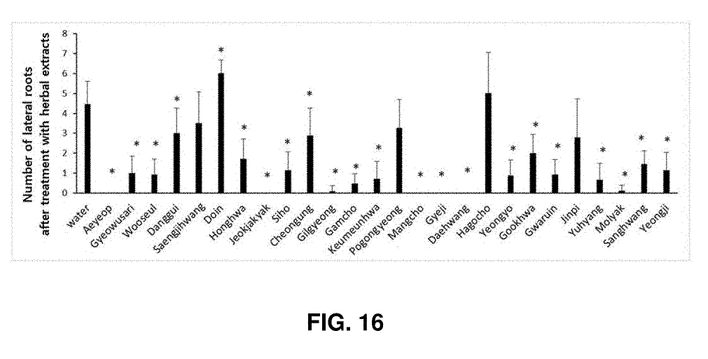

For making more quantitative analysis of the reaction to respective traditional herbal medicinal materials, the number of lateral roots were measured for 12 or more A. thaliana seeedlings in each treatment group. As shown in FIG. 16, it was seen that many traditional herbal medicinal materials inhibited generation of lateral roots although the levels of inhibition were not the same. However, it was shown that some traditional herbal medicinal materials, for example, hagocho, did not have a significant effect on generation of lateral roots, or, on the contrary, doin facilitated generation of lateral roots.

(5) Measuring Concentration of IR50

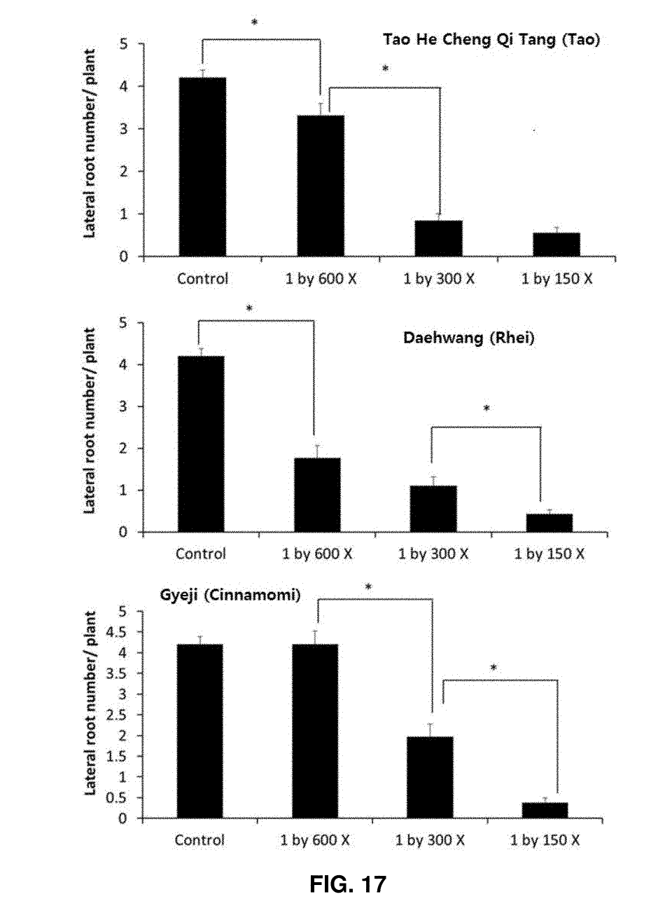

Exemplary medicinal materials and a drug which inhibit generation of lateral roots including Tao He Cheng Qi Tang, daehwang and gyeji were analyzed in more detail to find out the concentrations of crude extracts required to reduce the number of lateral roots to the half of control lateral roots. In conversion of respective crude extracts to the weight of powder obtained by freeze-drying it, Tao He Cheng Qi Tang was 24 mg/ml; daehwang was 30 mg/ml; and gyeji was 3 mg/ml. Based on the statistics, the concentrations of respective crude extracts required to inhibit generation of lateral roots to the half in terms of the number of lateral roots were very low, that is, 74 ug/ml, 77 ug/ml and 11 ug/ml, respectively, for Tao He Cheng Qi Tang, daehwang and gyeji as shown in FIG. 17. As described above, considering that A. thaliana roots respond to a very small quantity of crude extracts, it is seen that the plant response is a very unique and sensitive system.

(6) Responses of A. thaliana Seedlings to Other Traditional Herbal Medicinal Materials

6-1. Observation on Effect of Crude Ginseng Extract on Growth of A. thaliana Seedlings

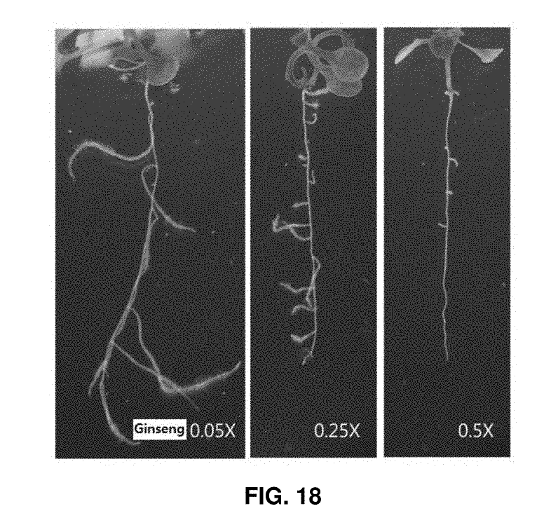

FIG. 18 is a photograph showing A. thaliana roots to illustrate response thereof to different concentrations of crude ginseng extract.

The result of A. thaliana seedlings growth in different concentrations of ginseng extract revealed overall root lengths were shorter as shown in FIG. 18 depending on the concentrations, and more lateral roots were generated just in a specific concentration. Specifically, in 0.05× which is a lower concentration, lateral roots developed and had dense root hairs. Although more lateral roots were generated in 0.25×, the root length were shorter. In 0.5× which was the highest concentration, the phenotype was that lateral root growth was rather inhibited and the primary roots were shorter.

The change of inhibited root length growth in the concentrations equal to or higher than 0.25× is markedly shorter roots than the control roots treated with water, implying that the ingredient of ginseng has a negative effect on A. thaliana growth. Considering that the positive effect in generation of lateral roots and inhibition of primary roots depends on the concentrations, it is regarded that such a response is based on the ingredient of ginseng. Such characteristics can be used as phenotypes, that is, a marker, to separate specific effective ingredients from ginseng.

6-2. Observation on Effect of Crude Jacho Extract on A. thaliana Seedlings

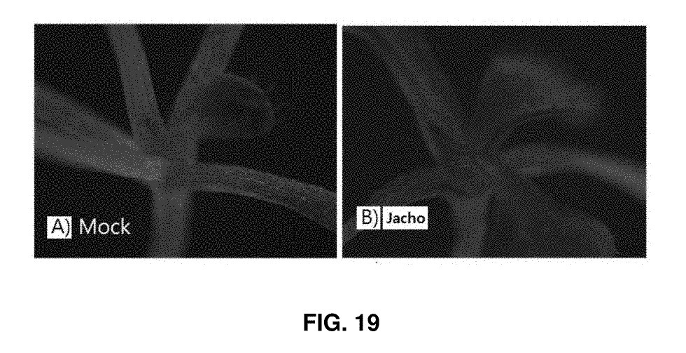

FIG. 19 is a photograph showing A. thaliana leave petiole of which color changes to reddish brown in response to the jacho extract.

In FIG. 19, A shows control A. thaliana leafe petiole and B shows changing color thereof after treatment with the crude jacho extract. It is seen that leaf petiole color changed from green in FIG. A to reddish brown in FIG. B. This is a unique response to the jacho extract. When the bioactive compound inducing this response is separated from jacho, it can be used as phenotypes, that is, a marker.

What is claimed is:

1. A method for discovering effective bioactive compounds, based on plant developmental biology, the method comprising screening candidates for developing a new drug or herbicide by establishing unique phenotypes shown in a plant as a marker when the plant is grown in media containing a specific compound.

2. The method of claim 1, wherein the plant is A. thaliana (Arabidopsis thaliana).

3. The method of claim 1 or 2, wherein the plant is one of wild type, mutant and transgenic plants.

4. The method of claim 3, wherein the transgenic plant is a plant genetically modified by introducing chimeric genes which combine any one of genes of targeted human diseases, human cancer-causing genes, cosmetic genes, herbicide-resistant genes, and other genes having approved functions with reporter genes.

5. A method for standardizing effective traditional herbal medicinal materials, based on plant developmental biology, the method comprising establishing unique phenotypes shown in a plant as a standard when the plant is grown in media containing an extract of traditional herbal medicinal material to grade traditional herbal medicinal materials.

6. The method of claim 5, wherein the plant is a transgenic plant genetically modified by introducing chimeric genes which combine a gene promoter response sensitively to an extract of specific traditional herbal medicinal material with reporter genes.

7. The method of claim 6, wherein the traditional herbal medicinal materials are graded by quantitatively analyzing the level of reporter gene expression induced by the traditional herbal medicinal material.
